(19) United States
(12) Patent Application Publication
(10) Pub. No.: US 2007/0197646 A1
(43) Pub. Date: Aug. 23, 2007

(54) SUBSTITUTED TARAXASTANES USEFUL FOR TREATING VIRAL INFECTIONS

(75) Inventors: Barton James BRADBURY, Wallingford, CT (US); Mingjun Huang, Potomac, MD (US)

Correspondence Address: CANTOR COLBURN, LLP, 55 GRIFFIN ROAD SOUTH, BLOOMFIELD, CT, 06002, US

(73) Assignee: ACHILLION PHARMACEUTICALS, INC., New Haven, CT (US)

(21) Appl. No.: 11/676,803

(22) Filed: Feb. 20, 2007

Related U.S. Application Data
(60) Provisional application No. 60/775,138, filed on Feb. 21, 2006.

(51) Int. Cl. A61K 31/203; C07C 61/29

(52) U.S. Cl. 514/559; 562/403

(57) ABSTRACT

Substituted taraxastanes useful for treating viral infections, are provided herein. Thus, in a first aspect, the invention provides compounds of Formula I

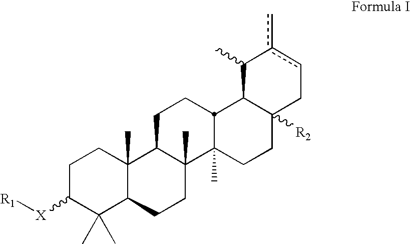

and the pharmaceutically acceptable salts thereof, wherein the variables R₁, R₂, and X are defined herein. The compounds described herein are thought to act by inhibiting retroviral maturation, including maturation of encapsulated retroviruses viruses, such as the HIV viruses, HIV-1 and HIV-2. Pharmaceutical compositions comprising such compounds of Formula I are included herein. Methods of using such compounds to treat human patients infected with an HIV virus and reducing the mortality of AIDS are also provided herein.

### CROSS REFERENCE TO RELATED APPLICATIONS

This application claims priority from U.S. provisional patent application No. 60/775,138, filed Feb. 21, 2006, which is hereby incorporated by reference ill its entirety.

### FIELD OF THE INVENTION

Substituted taraxastanes useful for treating viral infections are provided herein. Certain of these compounds act as highly active inhibitors of retroviruses, including HIV, and particularly HIV-1. Pharmaceutical compositions comprising such compounds are included in the invention. Methods of treating patients infected with an encapsulated virus, especially HIV, and reducing the mortality of viral diseases, such as AIDS, are also provided herein.

### BACKGROUND

Retroviruses are viruses that contain single-stranded RNA particles enveloped in a protein capsid. The retrovirus family consists of three groups: the spumaviruses such as the human foamy virus; the lentiviruses, such as the human immunodeficiency virus types 1 and 2, as well as visna virus of sheep; and the oncoviruses.

The retroviruses particle is composed of two identical RNA molecules. Each genome is a positive sense, single-stranded RNA molecule, which is capped at the 5′ end and polyadenylated at the 3′tail. The prototype C-type oncoviral RNA genome contains three open reading frames call gag, pol and env, bounded by regions that contain signals essential for expression of the viral genes. The gag region encodes the structural proteins of the viral capsid. The pol region encodes three viral enzymes including a protease, a reverse transciptase and an integrase. The env region specifies the glycoproteins of the viral envelope. In addition to these three open reading frames, the more complex genomes of the lentiviruses and the spumaviruses carry additional open reading frames, which encode regulatory proteins involved in the control of viral replication.

AIDS is a retroviral disease caused by HIV, a non-transforming human retrovirus belonging to the lentivirus family. Two genetically different but related forms of HIV, called HIV-1 and HIV-2, have been isolated from patients with AIDS. HIV-1 is the most common type associated with AIDS in the United States, Europe, and Central Africa, whereas HIV-2 causes a similar disease principally in West Africa.

Currently available drugs for the treatment of HIV infection target the reverse transciptase (RT) and HIV-1 protease (PR) enzymes, two of fifteen proteins encoded by the viral genome. These drugs are marginally effective when administered independently due to the rapid emergence of resistant strains that are selected under conditions of incomplete viral suppression. Sustained reductions in viral load can be achieved when RT and PR inhibitors are used in appropriate combinations (highly affective anti-retroviral therapy, HAART). But inadequate suppression due to poor compliance, resistance, and interactions with other drugs or diet is a significant problem that limits the effectiveness of HAART therapy for many patients and can lead to the spread of drug-resistant strains.

AIDS is characterized by profound immunosuppression that leads to opportunistic infections, secondary neoplasms and neurologic manifestations. In spite of the availability of HAART therapy, the mortality and morbidities associated with AIDS remain significant and unresolved by current therapies. New therapeutic compounds and methods that can reduce or ameliorate the adverse events and improve the clinical outcome of AIDS, for example, reducing mortality and improving the quality of life of those suffering from the disease, are needed. The invention fulfills this need by providing new therapeutic compounds useful for treating viral infections, including HIV infections, and provides further advantages, which are described herein.

### SUMMARY OF THE INVENTION

Compounds useful as inhibitors of viral replication are provided herein. Such compounds have antiviral activity, particularly against encapsulated viruses, including retroviruses including the HIV viruses HIV-1 and HIV-2. Compounds provided herein are generally substituted taraxastanes of Formula I and pharmaceutically acceptable salts thereof. Thus in a first aspect compounds and pharmaceutically acceptable salts of Formula I are provided herein.

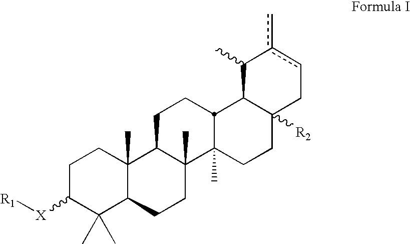

Wherein:
X is —CH₂—, NH, —O—, or a bond.

R₁ is hydrogen or R₁ is C₂-C₂₀carboxyalkanoyl; C₄-C₂₀carboxyalkenoyl; phosphonic acid C₂-C₂₀alkanoyl; or sulfonyl C₂-C₂₀alkanoyl; each of which is optionally substituted and wherein within each alkanoyl or alkenoyl a single methylene is optionally replaced by —O— or NR where R is hydrogen or C₁-C₄alkyl.

R₂ is —COOH, —CONH₂, —(CH₂)OR₃, —(CH₂)SR₃, —(CH2)ₓ(C═O)R₃, —(CH₂)ₓ(C═O)OR₃, —(CH₂)ₓ(C═O)NHR₃, or (CH₂)ₓ(C═O)NR₃R₄, where x is 0, 1, or 2.

R₃ and P₄ are independently C₁-C₂₀ alkyl, or C₂-C₂₀ alkenyl, each of which C₁-C₂₀ alkyl or C₂-C₂₀ alkenyl is optionally substituted and wherein within each C₂-C₂₀alkyl or C₂-C₂₀alkenyl one methylene is optionally replaced by a phenylene radical and one methylene is optionally replaced by —O—, —(C═O)NR₅—, —NR₅(C═O)—, or NR₅ where R₅ is independently hydrogen or C₁-C₄ alkyl.

Or, R₃ and R₄ may be taken together to form a 5- or 6-membered heterocycloalkyl ring, which is optionally substituted with halogen, hydroxyl, C₁-C₂alkyl, or C₁-C₂alkoxy.

A pharmaceutical composition, comprising a compound or salt of Formula I, in combination with at least one pharmaceutically acceptable carrier is also provided herein. The pharmaceutical composition may be formulated as an injectable fluid, ail aerosol, a cream, an oral liquid, a tablet, a gel, a pill, a capsule, a syrup, or a transdermal patch, or in another pharmaceutically efficacious form. The pharmaceutical composition may contain a compound of Formula I as the only active agent or may contain one or more additional active agents. The one or more additional active agents may be, for example, one or more additional compounds of Formula I, another anti-viral agent, or an immunostimulatory agent.

A method for inhibiting viral replication in cells, the method comprising contacting cells infected with a virus with a compound or salt of Formula I, in an amount sufficient to detectably inhibit viral replication in vitro, and thereby viral replication in the cells is provided herein.

Within certain embodiments provided herein the cells infected with a virus are present in a human.

A method of treating or preventing a viral infection in a human or non-human patient comprising providing a therapeutically effective amount of a compound or salt of Formula I to the patient is provided. The viral infection may be an infection with an encapsulated virus, such as a retrovirus, including the HIV viruses HIV-1 and HIV-2.

### BRIEF DESCRIPTION OF THE DRAWINGS

FIG. 1. ¹H NMR Spectra for compound 11 in CDCl₃ and DMSO.

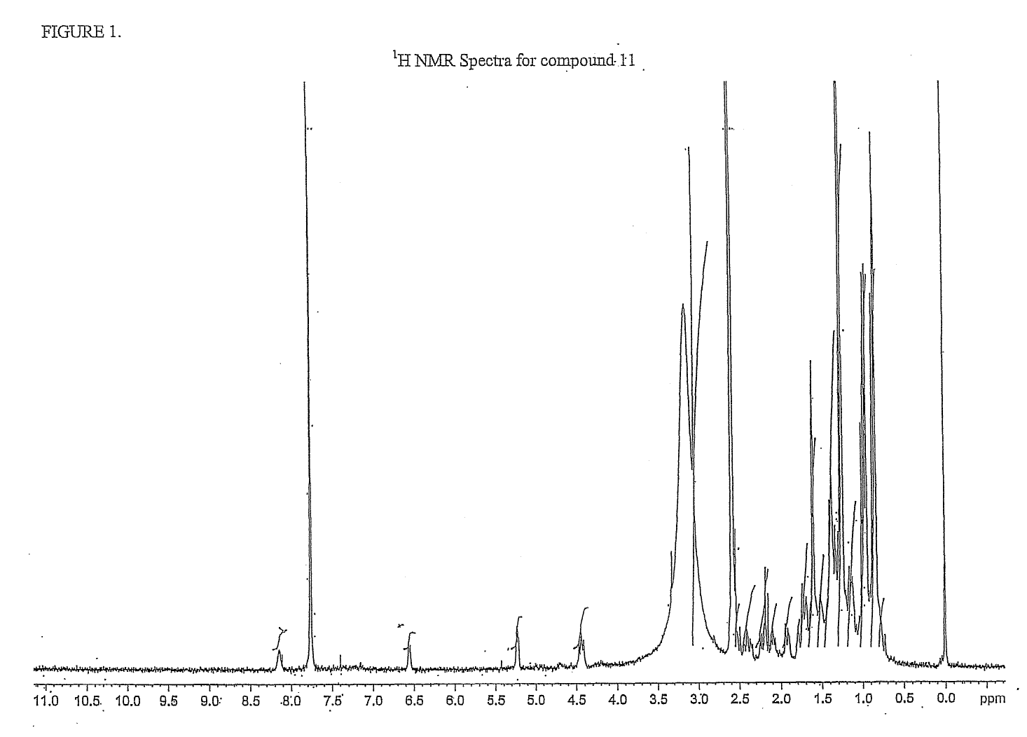

FIG. 2. IR Spectra for compound 11, 4 accumulations, resolution 4 cm⁻¹.

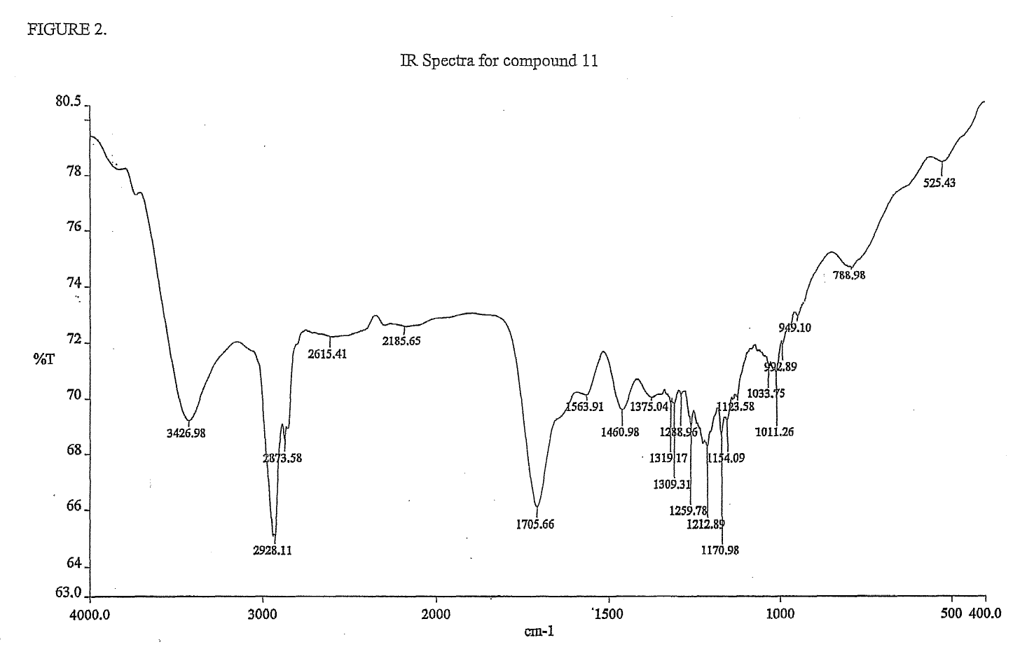

FIG. 3. ¹H NMR Spectra for compound 12 in CDCl₃ and DMSO.

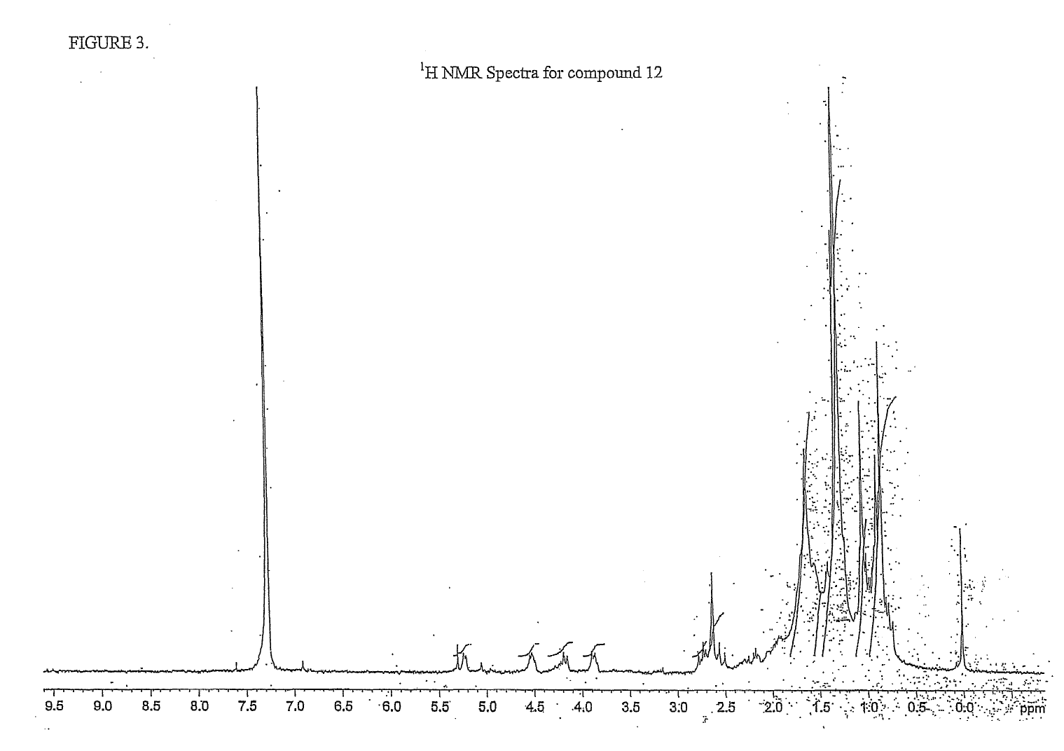

FIG. 4. IR Spectra for compound 12, 4 accumulations, resolution 4 cm⁻¹.

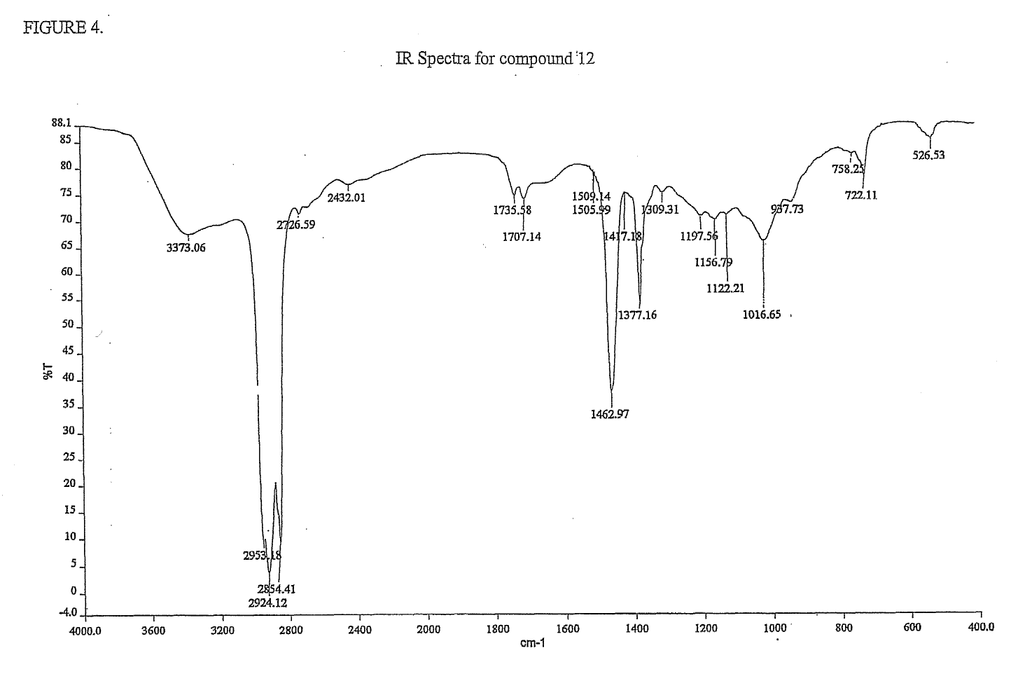

### DETAILED DESCRIPTION

As noted above, the present invention provides substituted taraxastanes and analogues thereof of Formula I. Such compounds may be used in vitro or in vivo, to inhibit activity of viruses, such as retroviruses, which include the HIV virus, and particularly the HIV-1 virus, and possess additional uses, as discussed in further detail below.

### Terminology

Compounds are generally described herein using standard nomenclature All pharmaceutically acceptable salt forms, hydrates, polymorphs, and prodrugs are included within the definition of any compound described herein. Where a compound exists in various tautomeric forms, a recited compound is not limited to any one specific tautomer, but rather is intended to encompass all tautomeric forms. Compound descriptions are intended to encompass compounds with all possible isotopes of atoms occurring in the compounds. Isotopes are those atoms having the same atomic number but different mass numbers. By way of general example, and without limitation, isotopes of hydrogen include tritium and deuterium and isotopes of carbon include ¹¹C, ¹³C, and ¹⁴C. Certain compounds are described herein using a general formula that includes variables (e.g., X and R₁). Unless otherwise specified, each variable within such a formula is defined independently of any other variable, and any variable that occurs more than one time in a formula is defined independently at each occurrence. In general, the variables (e.g. X, R₁, and R₂) may have any definition described herein that results in a stable compound.

An “active agent” is any compound, element, or mixture, that when administered to a patient alone or in combination with one or more other agents confers a therapeutic benefit on the patient. When the active agent is a compound solvates (including hydrates) of the free compound or salt, crystalline and non-crystalline forms, as well as various polymorphs or the compound are included. For example, an active agent can include optical isomers of the compound and pharmaceutically acceptable salts thereof either alone or in combination.

The term “substituted taraxastane(s)” encompasses all pharmaceutically acceptable salts, hydrates, and polymorphs of taraxastane compounds, for example of compounds of Formula I. “Substituted taraxastane(s)” also includes certain enantiomers, racemates, and stereoisomers of Formula I. “Substituted taraxastanes” includes stereoisomers (R and S forms) of compounds of Formula I in which the stereochemistry may be varied at bonds shown as wavy lines. The stereochemistry may also be varied at the positions indicated by the R₁, R₂, and X variables.

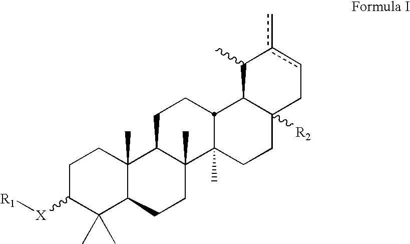

[symbol]

Indicates a bond that may be a double or single bond; the valence of the carbon atoms joined by the [symbol] bond must be satisfied and may not be exceeded. Within Formula I one bond indicated by [symbol] is a single bond and the other is a double bond. Thus Formula I encompasses the following two structures:

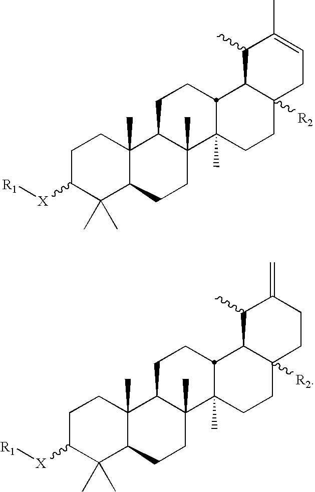

“Alkyl” refers to a straight chain or branched chain saturated aliphatic hydrocarbon having the indicated number of carbon atoms. For example a C₁-C₆alkyl group has from 1 to about 6 carbon atoms. Alkyl groups include groups having 1 to 20 carbon atoms (C₁-C₂₀alkyl) and from 1 to 10 carbon atoms (C₁-C₁₀alkyl), and from 1 to 4 carbon atoms (C₁-C₄alkyl) such as methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. An alkyl group may be bonded to an atom within a molecule of interest via any chemically suitable portion.

“Alkenyl” refers to straight or branched chain hydrocarbon groups, in which at least one unsaturated carbon-carbon double bond is present. Alkenyl groups include C₂-C₆alkenyl, and C₂-C₄alkenyl groups, which have from 2 to 6, or 2 to 4 carbon atoms, respectively, such as ethenyl, allyl, or isopropenyl.

“Alkoxy” means an alkyl group as described above attached via an oxygen bridge. Alkoxy groups include C₁-C₆alkoxy, and C₁-C₄alkoxy groups, which have from 1 to 6, or 1 to 4 carbon atoms, respectively. Alkoxy groups include, for example, methoxy, ethoxy, propoxy, isopropoxy, n-butoxy, sec-butoxy, tert-butoxy, n-pentoxy, 2-pentoxy, 3-pentoxy, isopentoxy, neopentoxy, hexoxy, 2-hexoxy, 3-hexoxy, and 3-methylpentoxy.

“Alkanoyl” indicates an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula CH₃(C═O)—.

“Alkylamino” refers to a secondary or tertiary amine having the general structure —NH(alkyl) or —N(alkyl)(alkyl), wherein each alkyl may be the same or different. Such groups include, for example, mono- and/or di-(C₁-C₆alkyl)amino groups, in which each alkyl is straight, branched or cyclic and may be the same or different and contains the indicated number of carbon atoms, for example from 1 to 6 carbon atoms or from 1 to 4 carbon atoms.

“Carboxyalkanoyl” indicates an alkyl group having a carboxylic acid (—COOH) group at one terminus and a keto (—(C═O)—) group at the other terminal end. The carboxyalkanoyl substitute is bound to the core molecule via a single covalent bond to the carbon of the keto group. The carbon atoms of the carboxylic acid group and keto group are included in the indicated carbon atoms. Thus a C₂carboxyalkanoyl is a group of the formula

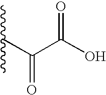

A group of the formula

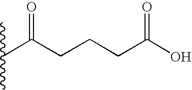

is an example of a C₅carboxyalkanoyl group.

“Carboxyalkenoyl” refers to an alkenyl group having a carboxylic acid group at one terminus and a keto (—(C═O)—) group at the other terminal end. “Phosphonic acid alkanoyl” refers to an alkanoyl group having a (—(P═O)(OH)₂ group at one terminus and a keto (—(C═O)—) group at the other terminal end. A “sulfonyl alkanoyl” has a sulfonyl (—SO₂H) group at one terminus and a keto (—(C═O)—) group at the other terminal end. For each of the carboxyalkenoyl, phosphonic acid alkanoyl, and sulfonyl alkanoyl groups described herein the point of attachment to the core molecule is a single covalent bond to the keto group carbon.

A “cycloalkyl” group is a fully saturated cyclic group containing carbon atoms as ring members. Cycloalkyl group include 3- to 7-membered cycloalkyl groups having a single saturated ring, e.g. cyclopropyl, cyclopentyl, and cyclohexyl.

The term “halogen” refers to fluorine, chlorine, bromine and iodine.

A “haloalkyl” is a branched or straight-chain alkyl group, substituted with 1 or more halogen atoms (e.g., “C₁-C₂haloalkyl” groups have from 1 to 2 carbon atoms). Examples of haloalkyl groups include, but are not limited to, mono-, di-, or tri-fluoromethyl; mono-, di-, or tri-chloromethyl; mono-, di-, tri-, tetra-, or penta-fluoroethyl; mono-, di-, tri-, tetra- or penta-chloroethyl; and 1,2,2,2-tetrafluoro-1-trifluoromethyl-ethyl. Typical haloalkyl groups are trifluoromethyl and difluoromethyl.

“Haloalkoxy” indicates a haloalkyl group as defined above attached through an oxygen bridge. “C₁-C₂haloalkoxy” groups have from 1 to 2 carbon atoms.

A dash (“-”) that is not between two letters or symbols is used to indicate a point of attachment for a substituent. For example, —CONH₂ is attached through the carbon atom.

A “heteroatom,” as used herein, is oxygen, sulfur, or nitrogen.

A “heterocycloalkyl” group is a heterocycle as described above, which is fully saturated. In certain embodiments preferred heterocycloalkyl groups are 5- to 7-membered heterocycloalkyl groups having a single saturated ring with 5 to 7 ring members, 1 or 2 ring members independently chosen from N, O, and S, with remaining ring members being carbon.

A “pharmaceutically acceptable salt” is an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, mesylic, benzene sulfonic, tosyllic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)ₙ—COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the compounds provided herein, including those listed by Remington's Pharmaceutical Sciences, 17th ed., Mack Publishing Company, Easton, Pa., p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in water or in an organic solvent, or in a mixture of the two; generally, the use of nonaqueous media, such as ether, ethyl acetate, ethanol, isopropanol or acetonitrile, is preferred.

A “prodrug” is a compound that may not fully satisfy the structural requirements of the compounds provided herein, but is modified in vivo, following administration to a patient, to produce a compound of Formula I, or other formula provided herein. For example, a prodrug may be an acylated derivative of a compound as provided herein. Prodrugs include compounds wherein hydroxy, amine or sulfhydryl groups are bonded to any group that, when administered to a subject, cleaves to form a free hydroxy, amino, or sulfhydryl group, respectively. Examples of prodrugs include, but are not limited to, acetate, formate and benzoate derivatives of alcohol and amine functional groups within the compounds provided herein. Prodrugs of the compounds provided herein may be prepared by modifying functional groups present in the compounds in such a way that the modifications are cleaved to the parent compounds.

A “substituent,” as used herein, refers to a molecular moiety that is covalently bonded to an atom within a molecule of interest. For example, a ring substituent may be a moiety such as a halogen, alkyl group, haloalkyl group or other group, that is covalently bonded to an atom (preferably a carbon or nitrogen atom) that is a ring member. Substituents or aromatic groups are generally covalently bonded to a ring carbon atom. The term “substitution” refers to replacing a hydrogen atom in a molecular structure with a substituent, such that the valence on the designated atom is not exceeded, and such that a chemically stable compound (i.e., a compound that can be isolated, characterized and tested for biological activity) results from the substitution.

The phrase “optionally substituted” indicates that such groups may either be unsubstituted or substituted at one or more of any of the available positions, typically 1, 2, 3, or 4 positions, by one or more suitable groups such as those disclosed herein.

Suitable groups that may be present on an “optionally substituted” position include, but are not limited to, e.g., halogen, cyano, hydroxyl, amino, intro, oxo, azido, alkanoyl (such as a C₂-C₆ alkanoyl group such as acyl or the like); carboxamido; alkylcarboxamide; alkyl groups, alkoxy groups, alkylthio groups including those having one or more thioether linkages, alkylsulfinyl groups including those having one or more sulfinyl linkages, alkylsulfonyl groups including those having one or more sulfonyl linkages, mono- and di-aminoalkyl groups including groups having one or more N atoms, all of the foregoing optional alkyl substituents may have one or more methylene group replaced by ail oxygen or —NH—, and have from about 1 to about 8, from about 1 to about 6, or from 1 to about 4 carbon atoms, cycloalkyl; phenyl; phenylalkyl with benzyl being an exemplary phenylalkyl group, phenylalkoxy with benzyloxy being an exemplary phenylalkoxy group; a saturated, unsaturated, or aromatic heterocyclic groups having 1 ring and one or more N, O or S atoms, e.g. pyridyl, pyrazinyl, pyrimidinyl, furanyl, pyrrolyl, thienyl, thiazolyl, triazinyl, oxazolyl, isoxazolyl, imidazolyl, tetrahydrofuranyl, tetrahydropyranyl, piperidinyl, morpholinyl, piperazinyl, and pyrrolidinyl. Any such groups having additional positions available for substitution may be further substituted, e.g. with substituents independently chosen from, e.g., amino, hydroxy, alkyl, alkoxy, halogen, haloalkyl, haloalkoxy, and mono- and di-alkylamino.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

“Providing” means giving, administering, selling, distributing, transferring (for profit or not), manufacturing, compounding, or dispensing.

The term “therapeutically effective amount” means an amount effective, when administered to a human or non-human patient, to provide any therapeutic benefit such as an amelioration of symptoms, e.g., an amount effective to decrease the symptoms of a viral infection, and preferably an amount sufficient to reduce the symptoms of an HIV infection. In certain circumstances a patient suffering from a viral infection may not present symptoms of being infected. Thus a therapeutically effective amount of a compound is also an amount sufficient to provide a positive effect on any indica of disease, e.g. an amount sufficient to prevent a significant increase or significantly reduce the detectable level of virus or viral antibodies in the patient's blood, serum, or tissues. A significant increase or reduction in the detectable level of virus or viral antibodies is any detectable change that is statistically significant in a standard parametric test of statistical significance, for example Student's T-test, where p<0.05.

### Inhibitors of Viral Replication

In addition to compounds and salts of Formula I, as described above, the invention provides compounds and salts of Formula I in which one or more of the following conditions are met:

Formula I includes compounds and salts thereof the formulae:

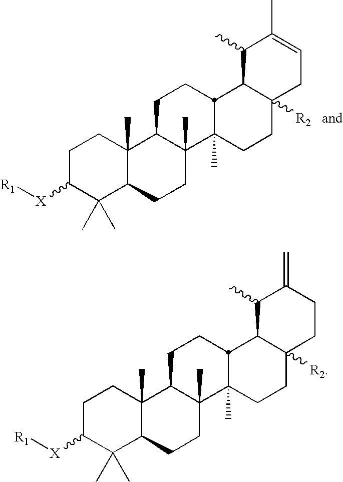

X is —O—.

(ii) R₁ is an optionally substituted C₂-C₂₀carboxyalkanoyl wherein within the alkanoyl a single methylene is optionally replaced by —O—.

(iii) R₁ is a C₄-C₁₆ carboxyalkanoyl group that is geminally substituted at the 3′ carbon atom.

(iv) R₁ has the formula —(C═O)CH₂CR′R″(CH₂)ₙCOOH where n is an integer of from 0 to 12 (or in certain embodiments n is an integer of from 0 to 4); R′ and R″ are each independently C₁-C₄alkyl, or R′ is hydrogen and R″ is C₁-C₄alkyl, or R′ and R″ are taken together to form 3- to 7-membered cycloalkyl ring or a 3- to 7-membered heterocycloalkyl ring having 1 or 2 heteroatoms independently chosen from N, O, and S.

(v) R₁ has the formula —(C═O)CH₂C(CH₃)₂(CH₂)ₙCOOH where n is 0 or 1.

(vi) R₁ has the formula —(C═O)CH₂O(CH₂)ₙCOOH where n is an integer of from 0 to 12.

(vii) R₁—X— is one of

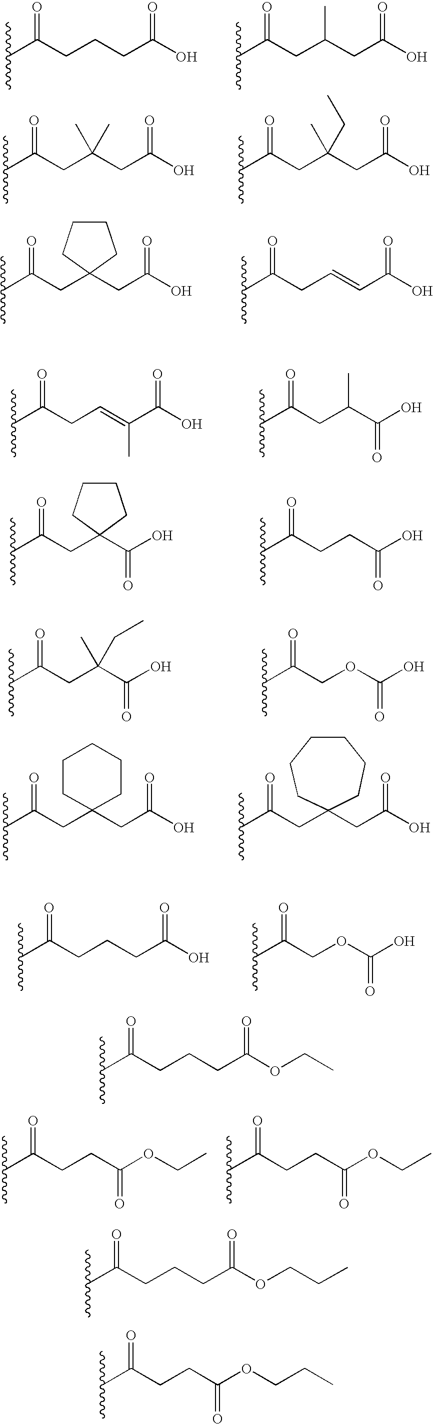

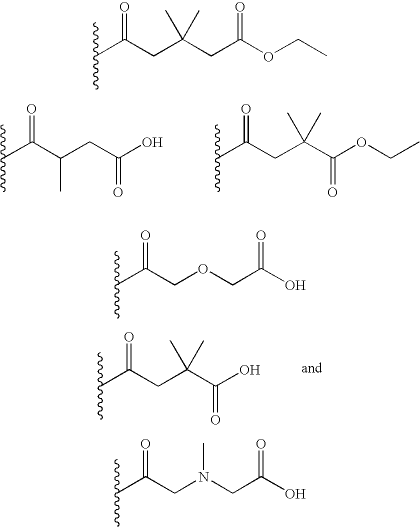

(viii) R₁ is

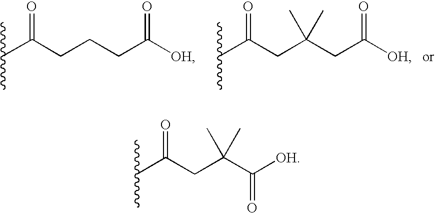

(ix) R₂ is a group of the formula (i)

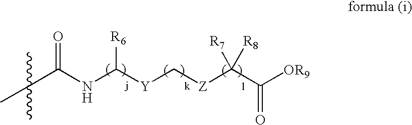

wherein: j, k, and l are integers from 0 to 16, wherein the sum of j, k, and l is from 4 to 16.

Y is a bond, —O—, —(C═O)NR₅—, —NR₅(C═O)—, or NR₅; and Z is a bond or a phenylene radical and R₆, R₇, R₈, and R₉ are independently hydrogen or C₁C₄alkyl; and the total number of methylene and methyl carbons in the group of formula (i) does not exceed 20.

(x) R₂ is —(C═O)NHR₃.

(xi) R₂ is —(C═O)NHR₃ and R₃ is an optionally substituted C₂-C₂₀alkyl wherein within the alkyl a single methylene is optionally replaced by —(C═O)NR₅—.

(xii) R₂ is —(C═O)NHR₃ and R₃ is terminally substituted with —COOH, —(C═O)OCH₃, or —CONH₂.

(xiii) R₂ is

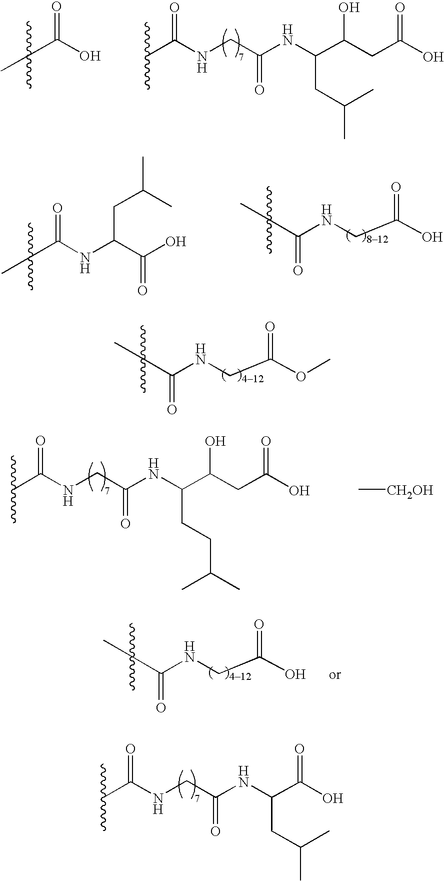

(xiv) R₂ is —COOH.

In some embodiments it is preferred that R₁ is not hydrogen unless R₂ is a group containing at least 2 carbon atoms or at least 3 carbon atoms.

Any of the above conditions for Formula I may be combined so long as a stable compound results.

### Pharmaceutical Compositions

Compounds and salts of Formula I can be administered as the neat chemical, but are usually combined with an adjuvant, diluent, excipient, or other ingredient.

Compounds of general Formula I may be administered orally, topically, as an ophthalmic solution, or by other means, in dosage unit formulations containing conventional non-toxic pharmaceutically acceptable carriers.

A pharmaceutical composition comprising a compound or salt of Formula I wherein the composition is formulated as an injectable fluid, an aerosol, a cream gel, a pill, a capsule, a tablet, a syrup, a transdermal patch, or an ophthalmic solution is provided herein.

In addition to the subject compound, the compositions of the invention may contain a pharmaceutically acceptable carrier, one or more compatible solid or liquid filler diluents or encapsulating substances, which are suitable for administration to an animal. Carriers must be of sufficiently high purity and sufficiently low toxicity to render them suitable for administration to the animal being treated. The carrier can be inert or it can possess pharmaceutical benefits of its own. The amount of carrier employed in conjunction with the compound is sufficient to provide a practical quantity of material for administration per unit dose of the compound.

Exemplary pharmaceutically acceptable carriers or components thereof are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cotton seed oil, sesame oil, olive oil, and corn oil; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

In particular, pharmaceutically acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil.

Oral formulations contain between 0.1 and 99% of a compound of the invention and usually at least about 5% (weight %) of a compound of the present invention. Some embodiments contain from about 25% to about 50% or from 5% to 75 % of a compound of invention.

### Liquids Formulations

Compounds of the invention can be incorporated into oral liquid preparations such as aqueous or oily suspensions, solutions, emulsions, syrips, or elixirs, for example. Moreover, formulations containing these compounds can be presented as a dry product for constitution with water or other suitable vehicle before use. Such liquid preparations can contain conventional additives, such as suspending agents (e.g., sorbitol syrup, methyl cellulose, glucose/sugar, syrup, gelatin, hydroxyethyl cellulose, carboxymethyl cellulose, aluminum stearate gel, and hydrogenated edible fats), emulsifying agents (e.g., lecithin, sorbitan monsoleate, or acacia), non-aqueous vehicles, which can include edible oils (e.g., almond oil, fractionated coconut oil, silyl esters, propylene glycol and ethyl alcohol), and preservatives (e.g., methyl or propyl p-hydroxybenzoate and sorbic acid).

Orally administered compositions also include liquid solutions, emulsions, suspensions, powders, granules, elixirs, tinctures, syrups, and the like. The pharmaceutically acceptable carriers suitable for preparation of such compositions are well known in the art. Oral formulations may contain preservatives, flavoring agents, sweetening agents, such as sucrose or saccharin, taste-masking agents, and coloring agents.

Typical components of carriers for syrups, elixirs, emulsions and suspensions include ethanol, glycerol, propylene glycol, polyethylene glycol, liquid sucrose, sorbitol and water. Syrups and elixirs may be formulated with sweetening agents, for example glycerol, propylene glycol, sorbitol or sucrose. Such formulations may also contain a demulcent.

### Suspensions

For a suspension, typical suspending agents include methylcellulose, sodium carboxymethyl cellulose, AVICEL RC-591, tragacanth and sodium alginate; typical wetting agents include lecithin and polysorbate 80; and typical preservatives include methyl paraben and sodium benzoate.

Aqueous suspensions contain the active material(s) in admixture with excipients suitable for the manufacture of aqueous suspensions. Such excipients are suspending agents, for example sodium carboxymethylcellulose, methylcellulose, hydropropylmethylcellulose, sodium alginate, polyvinylpyrrolidone, gum tragacanth and gum acacia; dispersing or wetting agents; naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol substitute, or condensation products of ethylene oxide with partial esters derived from fatty acids and hexitol anhydrides, for example polyethylene sorbitan substitute. The aqueous suspensions may also contain one or more preservatives, for example ethyl, or n-propyl p-hydroxybenzoate.

Oily suspensions may be formulated by suspending the active ingredients in a vegetable oil, for example peanut oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide palatable oral preparations. These compositions may be preserved by the addition of an anti-oxidant such as ascorbic acid.

### Emulsions

Pharmaceutical compositions of the invention may also be in the form of oil-in-water emulsions. The oily phase may be a vegetable oil, for example olive oil or peanut oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate.

### Tablets and Capsules

Tablets typically comprise conventional pharmaceutically compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules (including time release and sustained release formulations) typically comprise one or more solid diluents disclosed above. The selection of carrier components often depends on secondary considerations like taste, cost, and shelf stability.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methylcellulose phthalate, ethyl cellulose, Eudragit coatings, waxes and shellac.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

### Injectable and Parenteral Formulations

Pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be sterile injectable solution or suspension in a non-toxic parentally acceptable diluent or solvent, for example as a solution in 1,3-butanediol. Acceptable vehicles and solvents include water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid are useful in the preparation of injectables.

Compounds of Formula I may be administered parenterally in a sterile medium. Parenteral administration includes subcutaneous injections, intravenous, intramuscular, intrathecal injection, or infusion techniques. The drug, depending on the vehicle and concentration used, can either be suspended or dissolved in the vehicle. Advantageously, adjuvants such as local anesthetics, preservatives and buffering agents can be dissolved in the vehicle. In compositions for parenteral administration the carrier comprises at least about 90% by weight of the total composition.

### Topical Formulations

Compounds of the invention may be formulated for local or topical application, such as for topical application to the skin and mucous membranes, such as in the eye, in the form of gels, creams, and lotions and for application to the eye or for intracisternal or intraspinal application. Topical compositions of the present invention may be in any form including, for example, solutions, creams, ointments, gels, lotions, milks, cleansers, moisturizers, sprays, skin patches, and the like.

Such solutions maybe formulated as 0.01% −10% isotonic solutions, pH about 5-7, with appropriate salts. Compounds of the invention may also be formulated for transdennal administration as a transdermal patch.

Topical compositions containing the active compound can be admixed with a variety of carrier materials well known in the art, such as, for example, water, alcohols, aloe vera gel, allantoin, glycerine, vitamin A and E oils, mineral oil, propylene glycol, PPG-2 myristyl propionate, and the like.

Other materials suitable for use in topical carriers include, for example, emollients, solvents, humectants, thickeners and powders. Examples of each of these types of materials, which can be used singly or as mixtures of one or more materials, are as follows:

Emollients, such as stearyl alcohol, glyceryl monoricinoleate, glyceryl monostearate, propane-1,2-diol, butane-1,3-diol, mink oil, cetyl alcohol, iso-propyl isostearate, stearic acid, iso-butyl palmitate, isocetyl stearate, oleyl alcohol, isopropyl laurate, hexyl laurate, decyl oleate, octadecan-2-ol, isocetyl alcohol, cetyl palmitate, dimethylpolysiloxane, di-n-butyl sebacate, iso-propyl myristate, iso-propyl palmitate, iso-propyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, and myristyl myristate; propellants, such as propane, butane, iso-butane, dimethyl ether, carbon dioxide, and nitrous oxide; solvents, such as ethyl alcohol, methylene chloride, iso-propanol, castor oil, ethylene glycol monoethyl ether, diethylene glycol monobutyl ether, diethylene glycol monoethyl ether, dimethyl sulphoxide, dimethyl formamide, tetrahydrofuran; humectants, such as glycerin, sorbitol, sodium 2-pyrrolidone-5-carboxylate, soluble collagen, dibutyl phthalate, and gelatin; and powders, such as chalk, talc, fullers earth, kaolin, starch, gums, colloidal silicon dioxide, sodium polyacrylate, tetra alkyl ammonium smectites, trialkyl aryl ammonium smectites, chemically modified magnesium aluminum silicate, organically modified montmorillonite clay, hydrated aluminum silicate, fumed silica, carboxyvinyl polymer, sodium carboxymethyl cellulose, and ethylene glycol monostearate.

The compounds of the invention may also be topically administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

### Other Formulations

Other compositions useful for attaining systemic delivery of the subject compounds include sublingual, buccal and nasal dosage forms. Such compositions typically comprise one or more of soluble filler substances such as sucrose, sorbitol and mannitol, and binders such as acacia, microcrystalline cellulose, carboxymethyl cellulose and hydroxypropyl methylcellulose. Glidants, lubricants, sweeteners, colorants, antioxidants and flavoring agents disclosed above may also be included.

Compositions for inhalation typically can be provided in the form of a solution, suspension or emulsion that can be administered as a dry powder or in the form of an aerosol using a conventional propellant (e.g., dichlorodifluoromethane or trichlorofluoromethane).

### Additional Components

The compositions of the present invention may also optionally comprise an activity enhancer. The activity enhancer can be chosen from a wide variety of molecules that function in different ways to enhance antiviral effects of compounds of the present invention. Particular classes of activity enhancers include skin penetration enhancers and absorption enhancers.

Pharmaceutical compositions of the invention may also contain additional active agents chosen from a wide variety of molecules, which can function in different ways to enhance the antiviral or therapeutic effects of a compound of the present invention. These optional other active agents, when present, are typically employed in the compositions of the invention at a level ranging from about 0.01% to about 15%. Some embodiments contain from about 0.1% to about 10% by weight of the composition. Other embodiments contain from about 0.5% to about 5% by weight of the composition.

In all of the foregoing embodiments the compoumd of the invention can be administered alone or as mixtures, and the compositions may ftuther include additional drugs or excipients as appropriate for the indication.

### Packaged Formulations

The invention includes packaged pharmaceutical formulations. Such packaged formulations include a pharmaceutical composition containing one or more compounds or salts of Formula I in a container and optionally include instructions for using the composition to treat an animal (typically a human patient) suffering from a viral infection, such as an HIV infection, and including an HIV-1 or HIV-2 infection, or to prevent a viral infection in an animal. In certain embodiments the instructions are instructions for using the composition to treat a patient suffering from an HIV-1 infection.

### Methods of Treatment

The invention includes methods of preventing and treating viral infections by providing a therapeutically effective amount of one or more compounds of Formula I to an human or non-human patient at risk for a viral infection or suffering from a viral infection. The animal may be a fish, amphibian, reptile or bird, but is preferably a mammal. In many embodiments the animal is a human patient. Methods disclosed herein for treating viral infections including retroviral infections, for example lentivirus infections such as HIV infections, including HIV-1 and HIV-2 infections.

Exemplary viruses include:
Arenaviruses
Bunyaviridae, such as bunyaviruses, hanta viruses, nairovinuses, and phlebovirus
Coronaviridae, such as coronaviruses, including the SARS virus, and toroviruses
Cystoviridae
Filoviridae, including the Marburg virus and Ebola viruses
Flaviviridae, such as flavivirus, pestiviruses, and Hepatitis C virus. Flaviviruses include tick borne encephalitis, yellow fever and dengue fever virus.
Hepadnaviridae, such as Hepatitis B viruses
Herpesviridae, such as simpleviruses, varicelloviruses, and cytomegaloviruses
Orthomyxoviridae, such as influenza viruses
Paramyxoviridae, such as paramyxoviruses, morbilliviruses, and pneumonviruses
Picornaviruses, such as enteroviruses, poliovirus, rhinoviruses, hepatitis A virus, encephalomyocarditis virus, and aphthovirus
Retrovirus, including lentiviruses, Human spumavirus, and oncoviruses. Lentiviruses include HIV viruses, and
Togaviridae, including rubella virus.

Methods of preventing or treating HIV-1 infections and of treating AIDS in human patients are particularly included herein.

In some circumstances an effective amount of a compound of Formula I may be an amount sufficient to reduce the symptoms of the viral infection. Alternatively an effective amount of a compound of Formula I may be an amount sufficient to significantly reduce the amount of virus particles or antibodies against the virus detectable in a patient's tissues or bodily fluids.

The invention also provides a method of prevent transmission of HIV or other viruses between individuals. For example the invention provides a method of preventing HIV transmission from an infected pregnant woman to her fetus comprising providing a therapeutically effective amount of a compound of Formula I to the woman and/or fetus during pregnancy, or immediately prior to, during, or subsequent to childbirth.

Methods of treatment also include inhibiting viral replication, in vivo, in an animal infected with a virus, by administering a sufficient concentration of a compound of Formula I to inhibit viral replication in vitro. The virus may be a retrovirus, such as HIV, including HIV-1. In some embodiments the animal is a human patient infected with HIV-1 or suffering from AIDS. Methods of treatment also include reducing mortality of HIV-1 infection and/or AIDS by providing a therapeutically effective amount of a compound of Formula I to a human patient infected with HIV-1. Reduction in mortality may be any accepted means for measuring reduction in mortality or increased survival rates. For example a reduction in mortality may be an higher 2 year or 5 year survival rate in HIV-1 infected patients administered a compound of Formula I compared to HIV-1 infected patients not administered a compound of Formula I and not given any other treatment for HIV-1 infection. By “sufficient concentration” of a compound administered to the animal is meant the concentration of the compound available in the animal's system to needed prevent or combat the infection. Such a concentration by be ascertained experimentally, for example by assaying blood concentration of the compound, or theoretically, by calculating bioavailability. The amount of a compound sufficient to inhibit bacterial survival in vitro may be determined, for example, with the cytoprotection assay given in Example 4a, which follows.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active agent.

Frequency of dosage may also vary depending on the compound used and the particular disease treated. However, for treatment of most HIV-1 infections, a dosage regimen of 4 times daily or less is preferred and a dosage regimen of 1 or 2 times daily or less is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### Pharmaceutical Combinations

Provided herein are combinations for pharmaceutical use, comprising a compound of Formula I and a second active agent. The second active agent may contain a single active chemical entity or more than one active chemical entity. The second active agent may be an immunomodulatory compound, or an antiviral agent. In some embodiments the pharmaceutical composition comprises Formula I and a second active agent, wherein the second active agent is an immunomodulatory compound or an antiviral agent or a combination comprising one or more of the foregoing active agents, with the proviso that the second active agent is not interferon, a nucleoside or a nucleoside analog. For example, the anti-viral agent may be a tyrosine kinase inhibitor, a CCR5 inhibitor, a non-nucleoside reverse transcriptase inhibitor, a nucleoside reverse transcriptase inhibitor, a DNA polymerase inhibitor, a protease inhibitor, a fusion inhibitor, an integrase inhibitor, or an immunomodulatory compound. The second active agent may be, or a combination comprising one or more of the foregoing active agents. In one embodiment, the second active agent is one that may be administered once per day or even less frequently. In another embodiment the second active agent is D4T (ZERIT®, Bristol-Myers Squibb, also known as stanvudine), carbovir, acyclovir (ZOVIRAX®, GlaxoSmithKline), 3TC, FTC (emtricitabine), RACIVIR® (a mixture of emtricitabine and its positive enantiomer), interferon, ddI (VIDEX®), ddC (Zalcitabine), or L-(−)-FMAU, Famciclovir, or Penciclovir or Elvucitabine.

The second active agent may be a single chemical entity or may be comprised of more than one chemical entity. When the second active agent includes more than one active chemical entity the compound of Formula I may be prepared in a single dosage form with one or more of the active chemical entities and the other(s) may be administered separately. Alternatively all active chemical entities may be combined with the compound of Formula I in a single dosage form.

Combinations containing a compound of Formula I and one or more other active agents are included herein. Certain embodiments pertain to combination containing Elvucitabine, an additional nucleoside analog, and either a non-nucleoside reverse transcriptase inhibitor or a protease inhibitor. The non-nucleoside reverse transcriptase inhibitor may be, for example, efavirenz (SUSTIVA, Bristol-Myers Squibb, New York, N.Y.) or nevirapine (VIRAMUNE, Boehringer Ingelheim, Danbury, Conn.).

An immunomodulatory compound is any compound capable of modifying or regulating an immune function, Immunomodulatory compounds, in the context of HBV and HIV treatment, include, but are not limited to, glucocorticoids, thalidomide, alpha interferon, and its analogs, IL-2, and hematopoietins.

Glucocorticoids include, but are not limited to, hydrocortisone, cortisone, prednisone, prednisolone, methylprednisolone, triamcinolone, paramethasone, betamethasone, and dexamethasone.

Thalidomide analogs include, but are not limited to, ACTIMID and REVIMID (Celgene, Warren, N.J.).

Hematopoietins include, but are not limited to, hematopoietin-1 and hematopoietin-2, and also include other members of the hematopoietin superfamily such as the various colony stimulating factors (e.g. (e.g. G-CSF, GM-CSF, M-CSF), Epo, SCF (stem cell factor), various Interleukins (IL-1, IL-3, IL-4, IL-5, IL-6, IL-11, IL-12), LIF, TGF-beta, TNF-alpha) and other low molecular weight factors (e.g. AcSDKP, pEEDCK, thymic hormones, and minicytokines).

The second active agent may comprise a tyrosine kinase inhibitor. Tyrosine kinases are a class of enzymes that catalyze the transfer of the terminal phosphate of adenosine triphosphate to the phenolic hydroxyl group of a tyrosine residue present in a target protein. Tyrosine kinases play a critical role in signal transduction for several cellular functions including cell proliferation, carcinogenesis, apoptosis, and cell differentiation. Tyrosine kinase inhibitors have been found to have antiviral properties and may be used as the second active agent. Tyrosine kinases inhibitors include Imatinib mesylate (GLEEVEC or GLIVEC, Novartis), Gefitinib (IRESSA, Astra Zeneca), and erlotinib (TARCEVA, OSI Pharmaceuticals). Several other tyrosine kinase inhibitors are in phase II or phase III clinical trials. For example, AMN107 (Novartis Pharma AG) is slated to enter Phase II clinical trials for the treatment of chronic myeloid leukemia and sunitinib malate (also known as SU11248, brand name SUTENT, Pfizer) is in Phase II clinical trials for the treatment of cancer. Combinations of Elvucitabine and sunitinib malate and optionally AMN107 are within the scope of the invention.

The second active agent may comprise a CCR5 inhibitor. CCR5 is a receptor involved in HIV-1 fusion and entry. Tyrosines and negatively charged residues in the amino-terminal domain of CCR5 may be important for this function. Inhibitors of CCR5 include small molecules, peptides, chemokines and their derivatives, and monoclonal antibodies. CCR5 inhibitors currently being tested include GlaxoSmithKlines's GSK-873,140 (aplaviroc) and Schering Plough Corporation's SCH-417690 (vicriviroc). Other CCR5 inhibitors include 1-[(2,4-dimethyl-3-pyridinyl)carbonyl]-4-methyl-4-[3(S)-methyl-4-[1(S)-[4-(trifluoromethyl)phenyl]ethyl]-1-piperazinyl]-piperidine N1-oxide; SCH-C; SCH-D; TAK-779; UK-427,857 or marviroc (Pfizer), antibodies to CCR5; and combinations comprising one or more of the foregoing CCR5 inhibitors.

The second active agent may comprise an additional nucleoside reverse transcriptase inhibitor. Reverse transcriptase inhibitors interfere with the reverse transcriptase enzyme and prevent the virus from replicating. Suitable nucleoside reverse transcriptase inhibitors include lamivudine (EPIVIR, 3TC, GlaxoSmithKline), elvucitabine (Achillion), zidovudine (RETROVIR (AZT), (GlaxoSmithKline), lamivudine and zidovudine combination (COMBIVIR, GlaxoSmithKline), emtricitabine (EMTRIVA, Gilead), abacavir and lamivudine (EPZICOM, GlaxoSmithKline), zalcitabine (HIVID, Roche US Pharmaceuticals), abacavir, zidovudine, and lamivudine (TRIZIVIR, GlaxoSmithKline), tenofovir disoproxil fumarate and emtricitabine (TRUVADA, Gilead), didanosine (VIDEX, VIDEX EC (extended release), Bristol Myers-Squibb), stauvudine (ZERIT, Bristol Myers-Squibb), and abacavir (ZIAGEN, GlaxoSmithKline), acyclovir, ddI, ddC, and d4T. Combinations of nucleoside reverse transcriptase inhibitors may also be employed.

The second active agent may comprise a non-nucleoside reverse transcriptase inhibitor. Suitable non-nucleoside reverse transcriptase inhibitors include (S)-6-chloro-4-(cyclopropylethynyl)-1,4-dihydro-4-(trifluoromethyl)-2H-3,1-benzoxazin-2-one (efavirenz) (SUSTIVA, Bristol-Myers Squibb), 9-[(R)-2-[[bis[[(isopropoxycarbonyl)oxy]methoxy]phosphinyl]methoxy]propyl]adenine disoproxil fumarate (tenofovir) (VIREAD, Gilead), and delaviridine (RESCRIPTOR, Pfizer). Combinations of non-nucleoside reverse transcriptase inhibitors may also be employed.

The second active agent may comprise a protease inhibitor. Protease inhibitors interfere with a viral protease, an enzyme that cuts newly created protein chains into smaller proteins. Suitable protease inhibitors include, for example, amprenavir (AGENERASE, GlaxoSmithKline), fos-amprenavir (LEXIVA, GlaxoSmithKline), GW-433908 (prodrug of Amprenavir, Glaxo/Vertex), indinavir (CRIXIVAN, Merck), saquinavir (FORTOVASE, Roche), saquinavir mesylate (INVERASE, Hoffman-La Roche), nelfinavir (VIRACEPT, Pfizer), ritonavir (NORVIR, Abbott Laboratories), a blend of lopinavir and ritonavir (KALETRA, Abbott Laboratories), atazanavir (REYATAZ, Bristol-Myers Squibb), tipranavir (APTIVUS, Boehringer-Ingelheim), and combinations comprising one or more of the foregoing protease inhibitors.

The second active agent may comprise an integrase inhibitor. Integrase inhibitors prevent HIV from inserting its own genetic material into the host cell, so HIV cannot make new viruses. Several integrase inhibitors are currently being tested. These include JTK 303 from Gilead and Merck's L-870,810.

The second active agent may comprise a fusion inhibitor. Fusion inhibitors block HIV entry into cells by interfering with the fusion process of the virus and cell membrane. Certain fusion inhibitors block HIV entry into CD4 cells. One example of a suitable fusion inhibitor is enfuvirtide (FUZEON, Hoffman-La Roche and Trimeris).

The second agent may be hydroxyurea, a compound used to treat leukemia that acts by suppressing a host cell enzyme necessary for HIV reproduction, foscarnet, or phosphonoacetic acid.

### EXAMPLES

### Example 1

### Synthesis of Betulonic Acid from Betulinic Acid (Scheme 1)

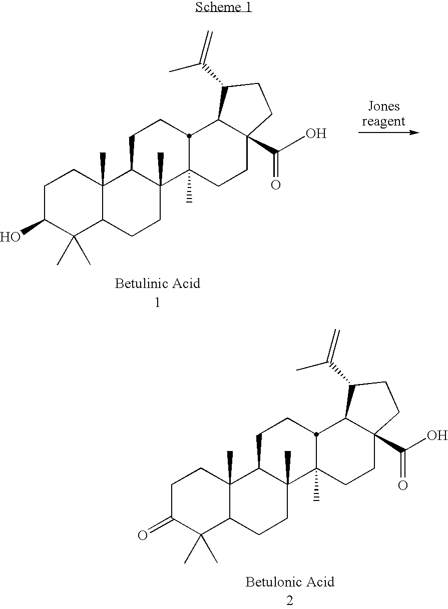

Freshly prepared Jones reagent (4.2 mL) is added to a stirring mixture of betulinic acid (2 g, 4.3 mmol) in acetone (1 L). After stirring for 10 minutes, the acetone is removed under reduced pressure until the volume is reduced to approximately 20% of the original volume (200 mL). Water is added (1 L) and the solution is filtered. The resulting solid is further purified by flash chromatography (80/20 hexane/ethyl acetate) to yield betulonic acid. Further purification by crystallization in ethyl acetate and hexane is sometimes necessary.

### Example 2

### Synthesis of Heterobetulinic Acid from Betulin (Scheme 2)

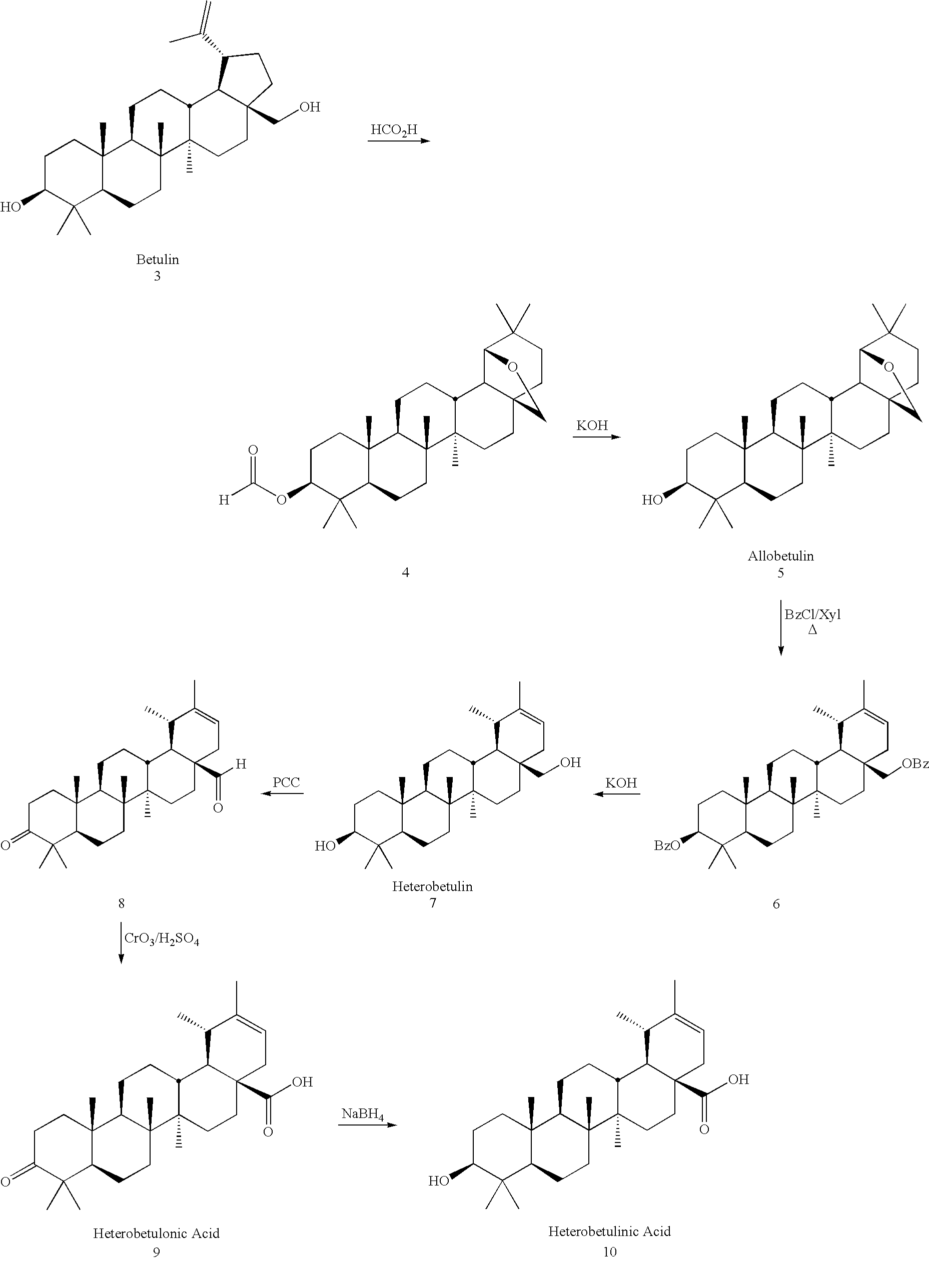

### Step 1. Preparation of Formic Allobetulin (4)

Betulin (3, 1.6 g, 3.62 mmol) is dissolved in formic acid (19.2 mL). The solution is refluxed for 2 hours. After cooling, ethanol (48 mL) is then added and the solution is stirred for 2 hours. The solution is filtered, and vacuum dried to obtain compound which is used in the next step without further purification.

### Step 2. Preparation of Allobetulin (5)

Formic allobetulin (4, 1.05 g, 2.24 mmol) is dissolved in toluene (70 ml) and 1N KOH/EtOH (8.6 mL), and the solution is refluxed for 1 hour. After cooling, the mixture is washed (using equal volumes) 3 times with water, one time with 1N HCl, and one time with water. The organic layer is dried with MgSO₄, filtered, and rotovapped, resulting in the crude product (5). The crude product is purified by silica gel column-chromatography.

### Step 3. Dibenzoylheterobetulin (6)

Allobetulin (5, 0.4 g, 0.90 mmol) is placed in a dry flask under argon. Dry diphenyl ether (2-4 mL) is added to the flask and the mixture is heated to dissolve the solid. Freshly distilled benzoyl chloride (0.4 mL, 3.2 equivalents) is added to the solution and the mixture is refluxed for 10 hours. The solution is cooled to room temperature, MeOH (6 mL) is added, and the mixture stirred overnight. A small amount of solid is collected, which is filtered and the filtrate concentrated to obtain the crude product as thick syrup. The crude product is purified by silica gel column chromatography (100-200 mesh) using hexane-ethyl acetate (1-2%) to obtain the pure dibenzoylheterobetulin as a white crystalline solid. Before the elution of the pure compound a small amount of diphenyl ether is also collected.

### Step 4. Preparation of Heterobetulin (7)

Dibenzoylheterobetulin (6, 0.4 g, 0.615 mmol) is saponified in toluene (67 ml) and 1N KOH/ethanol (10.5 mL) for two hours. The solution is washed 2 times with water, dried with NaSO4, filtered and rotovapped, resulting in a crude product (7). The crude product is purified by silica gel column chromatography (100-200 mesh) using hexane-ethyl acetate (5-7%) to obtain the pure as a white crystalline solid.

### Step 5. Preparation of Heterobetulonic Aldehyde (8)

Heterobetulin (0.15 g, 0.34 mmol) is added to CH₂Cl₂ (300 mL) and anhydrous sodium acetate (2.087 g, 25.4 mmol). PCC (0.547 g, 2.54 mmol) is dissolved in CH₂Cl₂ (150 mL) and added to the reaction. The reaction is stirred for 2 hours. Diethylether (300 mL) is added to the solution and the solution is filtered through Celite, then through neutral alumina, and the solvent removed under reduced pressure to yield 8 as a crude solid.

### Step 6. Preparation of Heterobetulonic Acid (9)

Heterobetulonic aldehyde (8, 50 mg, 0.114 mmol) is dissolved in acetone (10 mL), and freshly prepared Jones reagent (0.1 mL) is added. The reaction is stirred for 15 minutes. Water (15 mL) is added and the solution filtered giving 9 as a white solid. The crude product is purified by silica gel column chromatography (100-200 mesh) using hexane-ethyl acetate (7-8%) to afford the pure acid 9 as a white crystalline solid.

### Step 7. Heterobetulinic Acid (10)

Heterobetulonic acid (9, 30 mg, 0.066 mmol) is dissolved in THF (50 mL) and cooled in an ice bath. NaBH₄ (22 mg) is added to the solution. The solution is warmed to room temperature overnight. The reaction is quenched with 1 N HCl and the solvent removed under reduced pressure. The resulting residue is dissolved in chloroform and the solution washed with equal volumes of saturated NaCl solution 2 times, and once with water. The organic layer is dried with anhydrous sodium sulfate, filtered, and the solvent removed under reduced pressure to deliver 10 as an off-white solid.

### Example 3

### Synthesis of 2,2′-Dimethylsuccinate Ester of Terpenes

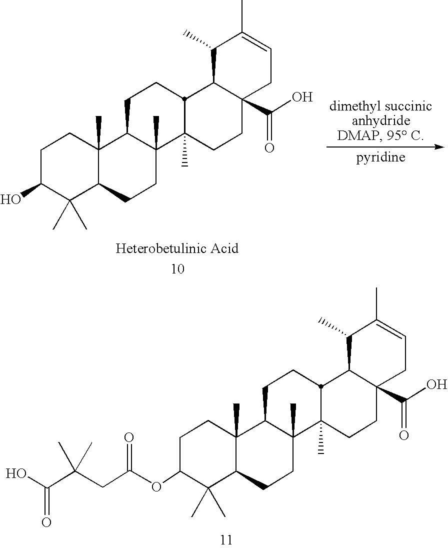

A mixture of betulinic/ursolic/oleanolic acids or heterobetulin (100 mg, 1.0 equivalant), dimethylamino pyridine (DMAP, 1.0 equivalent), 2,2′-dimethylsuccinic anhydride (10 equivalants) is refluxed in anhydrous pyridine (6 ml) for 18 hours at 110° C.

After the completion of the reaction, pyridine from the reaction mixture is completely removed in vacuo. The crude material is then taken into toluene and evaporated in vacuo 2-3 times to remove any traces of pyridine. The solid thus obtained is then triturated with n-hexane and then with ethyl acetate, which removed some of the top impurities in the supernatant liquid. TLC indicates that the solid thus obtained is pure enough to submit for the analysis.

### Example 4

### Additional Terpenes Derivatives

The following succinic acid ester derivatives and other derivatives of heterobetulinic acid are synthesized using the method illustrated in Example 3.

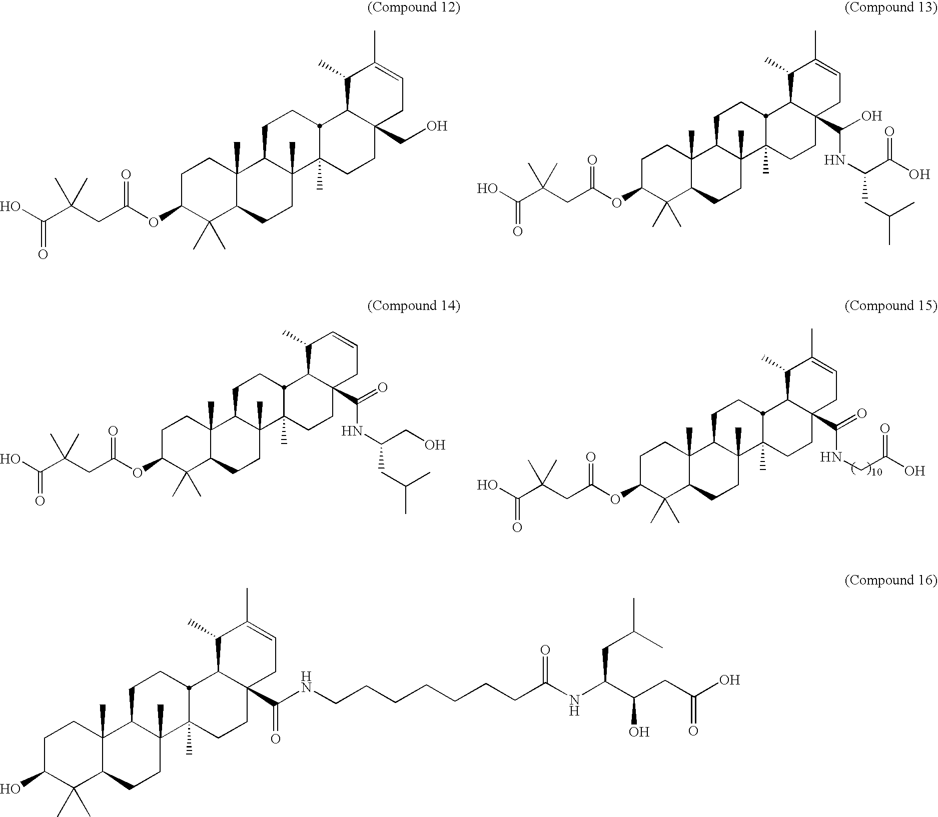

### Example 5

### Cell Viability and Virologic Replication Assays

Virology studies are preformed to assess the antiviral potency substituted heterobetulinic acids. The antiviral effect of these compounds is evaluated by parallel studies of inhibition of viral replication and cell viability. Viruses and cells for use in these assays may be obtained from the NIH AIDS Reagent Program.

5a. Determination of Antiviral Effect and Assessment of Cytotoxicity

Compounds of Formula I are tested in an in vitro cytoprotection assay. The cytoprotection assay utilizes a laboratory-adapted RF strain of HIV-1 and CEM-SS cells or the NL4/3 (Accession number M19921) strain of HIV-1 and MT4 cells (catalog #120 from the NIH AIDS Reagent Program).

CEM-SS Cells are well mixed and counted, and resuspended in RPMI 1640 media with 10% heat inactivated Fetal Bovine Sertun 2 mm L-glutamine, 100 U/ml penicillin, 100 μg/ml streptomycin, and 10 μg/ml gentamycin. Serial or half-serial dilutions of test compound in RPTI 1640 are prepared (5 dilutions for a typical 6-point curve). Typically half log dilutions of compound, at concentrations from 100 μM to 0.3 μM, are used for a 6 point cell viability curve. 100 μl test compound or control compound, such as 0.01 μM AZT, are pipetted into test wells of a 96-well plate. 50 μl CEM-SS cells and 50 μl virus of known MOI are added to each compound containing well. Reagent background wells containing only media and negative control wells containing 100 μl RPMI 1640 media, and 50 μl virus are also prepared.

Plates are incubated at 37° C. in a humidified CO₂ incubator for six days. Following incubation of the HIV-1 infected cells with the compound cell viability is assessed using a calorimetric assay of metabolic activity, such as an XTT, MTT or WST-1 assay, or a BrdU incorporation assay. Cytotoxicity of the compound is determined in parallel by measurement of the viability of mock-infected cells treated with the compound. Uninfected cells without compounds are used for a 100% viability control. Infected cells to which no compound is added provide a value for 100% of cell death due to viral infection. The number of surviving or viable cells is quantified by a tetrazolium-based colorimetric method using the CellTiter96 reagent (Promega, Madison, Wis.).

5b. Inhibition of Early Phase Virus Replication:

The effect of compounds on the early phase virus replication, from virus particle attachment through early gene, Tat expression, are evaluated using a CD4-positive LTR-β-galatosidase-expressing HeLa (MAGI) cell indicator line. This assay, described previously to assess the level of β-gal expression, has been modified and adopted in our laboratory. β-gal levels are quantified by photon detection assay, with virus infected cells that are not treated with a capsid formation inhibitor giving the highest level of expression. Effective capsid formation inhibitors reduce β-gal expression by 50% or more in the concentration range tested, and in many instances by more than 90%.

MAGI Cells are trypsinization and counted using the trepan blue exclusion method, and resuspended at a pre-determined concentration (usually 2×10⁴ cells/well) in 10% FBS complete RPMI 1640. Cells are incubated 37° C. overnight.

The viruses are diluted to a pre-determined MOI/well, typically 0.01 to 1, in Pre-MAGI media plus 20 μg/well DEAE-dextran.

The assay plates are set up as follows.

Each drug is tested at 6 doses and in triplicate. Typically half log dilutions of compound, at concentrations from 100 μM to 0.3 μM, are used for a 6 point curve.

Each plate includes the following control wells:

Virus control, virus and 20 ug/mL Dextran Sulfate, in a total of 200 uL Pre-MAGI media plus 20 ug/mL DEAE-dextran;

Cell control, cells in 200 uL Pre-MAGI media plus 20 ug/mL DEAE-dextran without drug or virus; and

Positive control, cells in 100 uL/well Pre-MAGI media plus 20 ug/mL DEAE-dextran, 50 μL prepared/diluted virus, and 50 μL of a compound of known activity.

Wells containing test compound are prepared by removing media from the cells and replacing with 100 uL/well Pre-MAGI media plus 20 ug/mL DEAE-dextran. 50 uL of 4×concentration test compound is added to each well. Add 50 uL of prepared/diluted virus is added per well. The plate(s) are incubated at 37° C. for 48 hours.

Following the 48 hour incubation period, the Gal-Screen Reagents are removed from 4° C. and allowed to warn to room temperature before using. Once the reagents have reached room temperature, the Gal-Screen Substrate is diluted 1:25 with Gal-Screen Buffer A or B (40 μL for every 960 μL).

100 μL of supernatant from all wells including Test Compound Wells, Cell Controls, Virus Controls and Positive Controls is removed and discarded. 100 uL of the Gal-Screen Reagent (Applied Biosystems, Foster City, Calif.) is added per well.

Plates are incubated at room temperature (25° C.) for 60-90 minutes or until constant light emission is reached. Plate(s) are read in a luminometer and measured for 0.1-1.0 sec/well. Compound 11 was tested in this assay and found to exhibit an EC50 of less than 2 micromolar at a MOI of 0.00125. Compound 12 was tested in this assay and found to exhibit an EC50 of less than 100 nM at a MOI of 0.00125. Neither compound was cytotoxic.

1. A compound having the formula 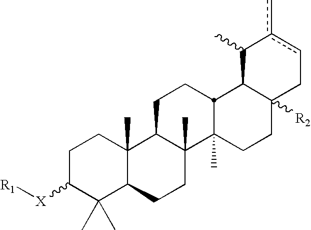
or a pharmaceutically acceptable salt thereof, wherein
X is —CH₂—, NH, —O—, or a bond; and
R₁ is hydrogen, or
R₁ is C₂-C₂₀carboxyalkanoyl; C₄-C₂₀carboxyalkenoyl; phosphonic acid C₂-C₂₀alkanoyl; or sulfonyl C₂-C₂₀alkanoyl; each of which is optionally substituted and wherein within each alkanoyl or alkenoyl a single methylene is optionally replaced by —O— or NR where R is hydrogen or C₁-C₄alkyl;
R₂ is —COOH or —CONH₂; or
R₂ is —(CH₂)OR₃, —(CH₂)SR₃, —(CH₂)ₓ(C═O)R₃, —(CH₂)ₓ(C═O)OR₃, —(CH₂)ₓ(C═O)NHR₃, or —(CH₂)ₓ(C═O)NR₃R₄, where x is 0, 1, or 2; and
R₃ and R₄ are independently C₁-C₂₀ alkyl, or C₂-C₂₀ alkenyl, each of which C₁-C₂₀ alkyl or C₂-C₂₀ alkenyl is optionally substituted and wherein within each C₂-C₂₀alkyl or C₂-C₂₀alkenyl one methylene is optionally replaced by a phenylene radical and one methylene is optionally replaced by —O—, —(C═O)NR₅—, —NR₅(C═O)—, or NR₅ where R₅ is hydrogen or C₁-C₄ alkyl, or
R₃ and R₄ may be taken together to form a 5- or 6-membered heterocycloalkyl ring, which is optionally substituted with one or more halogen, hydroxyl, C₁-C₂alkyl, or C₁-C₂alkoxy.

2. A compound or salt of claim 1 having the formula

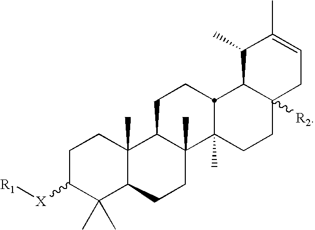

3. A compound or salt of claim 1 wherein X is —O—.

4. A compound or salt of claim 3 wherein
R₁ is an optionally substituted C₂-C₂₀carboxyalkanoyl wherein within the alkanoyl a single methylene is optionally replaced by —O—.

5. A compound or salt of claim 3, wherein R₁ is a C₄-C₁₆ carboxyalkanoyl group that is geminally substituted at the 3′ carbon atom.

6. A compound or salt of claim 3, wherein
R₁ has the formula —(C═O)CH₂CR′R″(CH₂)ₙCOOH where n is an integer of from 0 to 12;
R′ and R″ are each independently C₁-C₄alkyl, or
R′ is hydrogen and R″ is C₁-C₄alkyl, or
R′ and R″ are taken together to from 3- to 7-membered cycloalkyl ring or a 3- to 7-membered heterocycloalkyl ring having 1 or 2 heteroatoms independently chosen from N, O, and S.

7. A compound or salt of claim 6, wherein n is an integer of from 0 to 4.

8. A compound or salt of claim 6, wherein R′ and R″ are both methyl and n is 0 or 1.

9. A compound or salt of claim 3, wherein
R₁ has the formula —(C═O)CH₂O(CH₂)ₙCOOH where n is an integer of from 0 to 12.

10. A compound or salt of claim 1 wherein R₁—X— is one of

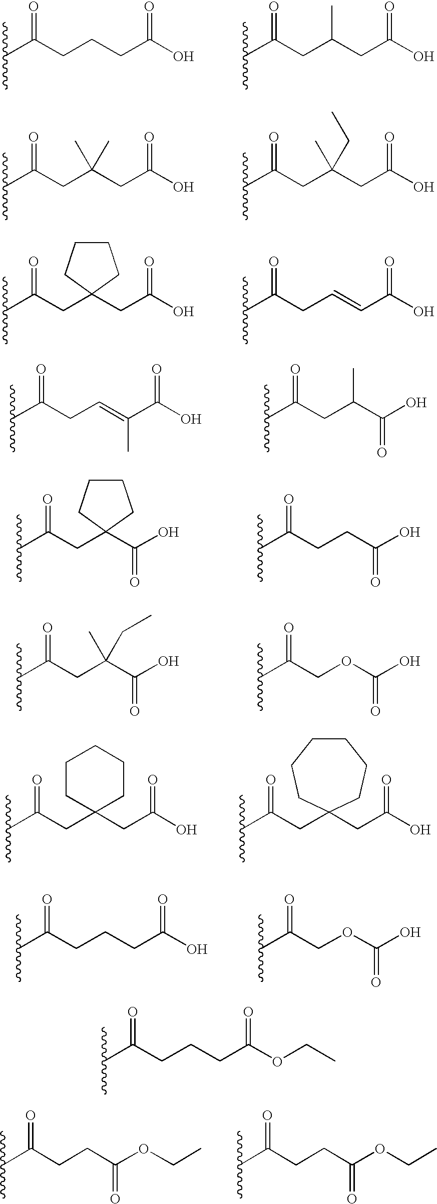

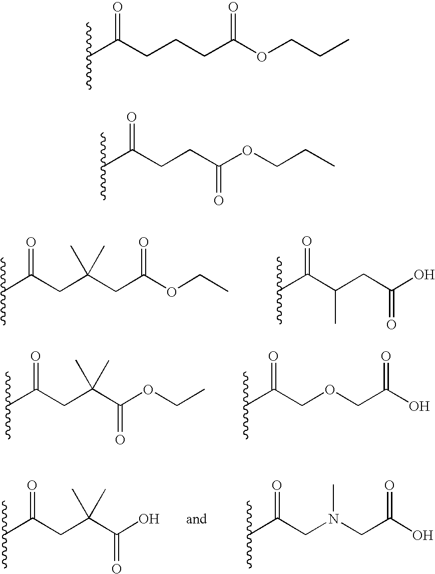

11. A compound or salt of claim 3, wherein R₁ is

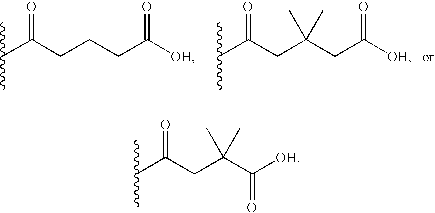

12. A compound or salt of claim 1 wherein R₂ is a group of the formula (i) 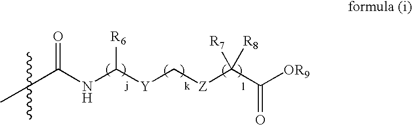
wherein
j, k, and l are integers from 0 to 16, wherein the sum of j, k, and l is from 4 to 16;
Y is a bond, —O—, —(C═O)NR₅—, —NR₅(C═O)—, or NR₅;
Z is a bond or a phenylene radical;
R₆, R₇, R₈, and R₉ are independently hydrogen or C₁-C₄alkyl; and the total number of methylene and methyl carbons in the group of formula (i) does not exceed 20.

13. A compound or salt of claim 1, wherein R₂ is —(C═O)NHR₃.

14. A compound or salt of claim 13 wherein R₃ is an optionally substituted C₂-C₂₀alkyl wherein within the alkyl a single methylene is optionally replaced by —(C═O)NR₅—.

15. A compound or salt of claim 13, wherein R₃ is terminally substituted with —COOH, —(C═O)OCH₃, or —CONH₂.

16. A compound or salt of claim 1, wherein R₂ is

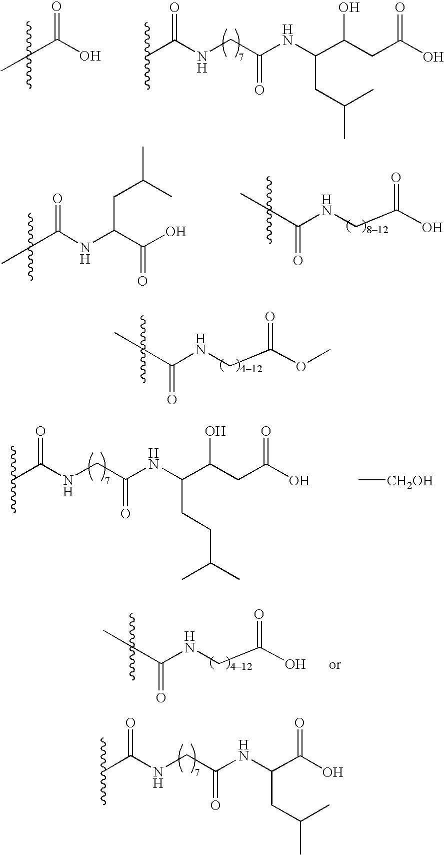

17. A pharmaceutical composition, comprising a compound or salt of claim 1, in combination with at least one pharmaceutically acceptable carrier.

18. The pharmaceutical composition of claim 17 additionally comprising one or more other active agents; wherein the active agent is an anti-viral agent, an immunostimulatory agent, or a combination of the foregoing.

19. The pharmaceutical composition of claim 18, wherein the anti-viral agent is zidovudine, lamivudine, zalcitabine, stavudine, didanosine, tenofovir, abacavir, nevirapine; delavirdine, emtricitabine, efavirenz, saquinavir, ritonavir, indinavir, nelfinavir, lopinavir, amprenavir, atazanavir, enfuvirtide, hydroxyurea, interleukin-2, amantadine, alpha-interferon, beta-interferon, gamma-interferon, rifampin, ribavirin, foscarnet, elvucitabine, phosphonoacetic acid, acyclovir, gancyclovir, or a combination of one or more of the foregoing.

20. The pharmaceutical composition of claim 17, wherein the composition is formulated as an injectable fluid, an aerosol, a cream, an oral liquid, a tablet, a gel, a pill, a capsule, a syrup, or a transdermal patch.

21. A method for inhibiting viral replication in cells, the method comprising contacting cells infected with a virus with a compound or salt of claim 1, in an amount sufficient to detectably inhibit viral replication in vitro, and thereby inhibiting viral replication in the cells.

22-25. (canceled)

26. A method of treating a patient infected with a retrovirus or susceptible to infection by a retrovirus comprising providing a therapeutically effective amount of a compound or salt of claim 1 to the patient.

27. The method of claim 26, wherein the patient is a human patient.

28. The method of claim 26 or 27 wherein the retrovirus is HIV.

29. The method of claim 28 wherein the retrovirus is HIV-1.

30. A method of reducing mortality of HIV-1 infection comprising providing a therapeutically effective amount of a compound of claim 1 to a human patient infected with HIV-1.
